(19) United States
(12) Patent Application Publication
(10) Pub. No.: US 2006/0155328 A1
(43) Pub. Date: Jul. 13, 2006

(54) Optimized suture braid

(75) Inventor: Seth A. Foerster, San Clemente, CA (US)

Correspondence Address: Donald E. Stout;Stout, Uxa, Buyan & Mullins, LLP, Suite 300, 4 Venture, Irvine, CA, 92618, US

(73) Assignee: Opus Medical, Inc., San Juan Capistrano, CA (US)

(21) Appl. No.: 10/803,455

(22) Filed: Mar. 18, 2004

Related U.S. Application Data
(60) Provisional application No. 60/455,843, filed on Mar. 18, 2003.

(51) Int. Cl. A61L 17/00

(52) U.S. Cl. 606/228

(57) ABSTRACT

A high strength abrasion resistant surgical suture material with industry standard knot tying characteristics and color marking characteristics includes a core formed of a plurality of twisted fibers of a first material, surrounded by a braided cover made from fibers of a second material different than the first material. The first material is preferably ultrahigh molecular weight polyethylene and the second material is preferably a polymeric material having good knot-tying characteristics.

This application claims the benefit of U.S. provisional patent application No. 60/455,843, filed on Mar. 18, 2003, the entire disclosure of which is incorporated herein by this specific reference.

### BACKGROUND OF THE INVENTION

The present invention relates to high strength surgical suture materials, and more particularly to braided suture blends of ultrahigh molecular weight polyethylene and polyester. The composite sutures have high tensile strength as well as excellent knot tying characteristics.

### DESCRIPTION OF THE RELATED ART

Strength, particularly tensile strength, is an important consideration in any surgical suture material. One of the strongest materials currently formed into elongated strands is an ultrahigh molecular weight long chain polyethylene (UHMWPE), typically used for fishing line and the like, which is sold under the trade names such as Dyneema® or Spectra®. However, this material, while much stronger than ordinary surgical suture, does not have acceptable knot tying characteristics for use in surgical applications because of its low frictional coefficient. Additionally, this material only comes in one color, making multiple suture distinction difficult, especially arthroscopically.

Current braided suture technology allows for acceptable knot tying characteristics through use of the material properties afforded by silicone-coated polyester. Polyester also comes in a variety of colors making it easy for the manufacturer to assemble many color patterns and color schemes to assist the surgeon in sorting out the many sutures used in a procedure.

Suture knot holding characteristics are a function of the suture's ability to frictionally lock to itself within the knot. The magnitude of this friction is determined by the coefficient of friction of the material used to form the suture, as well as the geometry of the suture-to-suture interface within the knot. Polyester's relatively high coefficient of friction has made it the material of choice within the industry, while the standard interface in the industry is woven braid upon woven braid. The individual elements of the braid in this crossed up interplay leave small round elements laying lo across each other, producing point contacts between the elements. Point contacts result in relatively low friction, and thus result in less than optimum knot tying abilities.

Accordingly, there exists a need for improved suture materials having high strength and excellent knot tying characteristics.

### SUMMARY OF THE INVENTION

The present invention advantageously provides a suture strand having high tensile strength as well as clinically acceptable knot-tying characteristics. Briefly, the suture strand comprises a core formed of a plurality of core fibers of a first material, surrounded by a cover including a plurality of cover fibers made of a second material different than the first material. Preferably, the first material is a high tensile strength, high tenacity material such as ultrahigh molecular weight long chain polyethylene, and the second material is a material having good knot-tying characteristics, such as a polymer selected, for instance, from the group consisting of PET, polyester, coated urethanes, and mixtures thereof.

The core may consist of a single core element comprising a plurality of core fibers that have been twisted together to form a bundle. Each fiber within the bundle itself comprises of a plurality of core filaments of the first material. The number of fibers in a bundle, as well as the cross-sectional shape of the bundle, may be varied to form different embodiments of the invention.

Alternatively, the core may comprise a sub-core surrounded by an outer ring. The sub-core may consist of a single core fiber, or it may comprises a plurality of core fibers arranged in a twisted bundle. The outer ring comprises an annular woven braid of core fibers. The number and cross-sectional shape of the core fibers in the sub-core may be varied to form different embodiments of the invention.

Advantageously, the filaments in both the core fibers and the cover fibers are circular in cross-section. The core filaments are preferably larger in diameter than the cover filaments. In embodiments having a sub-core and an outer ring, the filaments in the sub-core may be larger than or the same size as the filaments in the outer ring.

More particularly, in one aspect of the invention there is provided a suture strand suitable for use as a suture or ligature, which comprises a core including a plurality of core fibers made of a first material, and a cover surrounding the core, wherein the cover includes a plurality of cover fibers made of a second material different than the first material.

In another aspect of the invention, there is provided a suture strand suitable for use as a suture or ligature, which comprises a core including a plurality of core fibers consisting solely of a first material, and a cover surrounding the core, wherein the cover includes a plurality of cover fibers consisting solely of a second material different than the first material.

Additional aspects and advantages of the present invention are set forth in the following description and claims, particularly when considered in conjunction with the accompanying drawings in which like parts bear like reference numerals.

### BRIEF DESCRIPTION OF THE DRAWINGS

FIG. 1 is a cross-sectional view of a suture strand according to the present invention;

FIG. 2 is a cross-sectional view of a suture strand according to an alternate embodiment of the invention;

FIG. 3 is a perspective view, partially broken away, of the suture strand of FIG. 1;

FIG. 4 is a perspective view, partially broken away, of the suture strand of FIG. 2;

FIG. 5 is a cross-sectional view of a suture strand according to another embodiment of the invention;

FIG. 5 is a cross-sectional view of a suture strand according to yet another embodiment of the invention;

FIG. 7 is a perspective view, partially broken away, of the suture strand of FIG. 5;

FIG. 8 is a perspective view, partially broken away, of the suture strand of FIG. 6;

FIG. 9 is a cross-sectional view of a suture strand according to still another embodiment of the invention;

FIG. 10 a cross-sectional view of a suture strand according to yet still another embodiment of the invention;

FIG. 11 is a perspective view, partially broken away, of the suture strand of FIG. 9; and

FIG. 12 is a perspective view, partially broken away, of the suture strand of FIG. 10.

### DESCRIPTION OF THE PREFERRED EMBODIMENTS

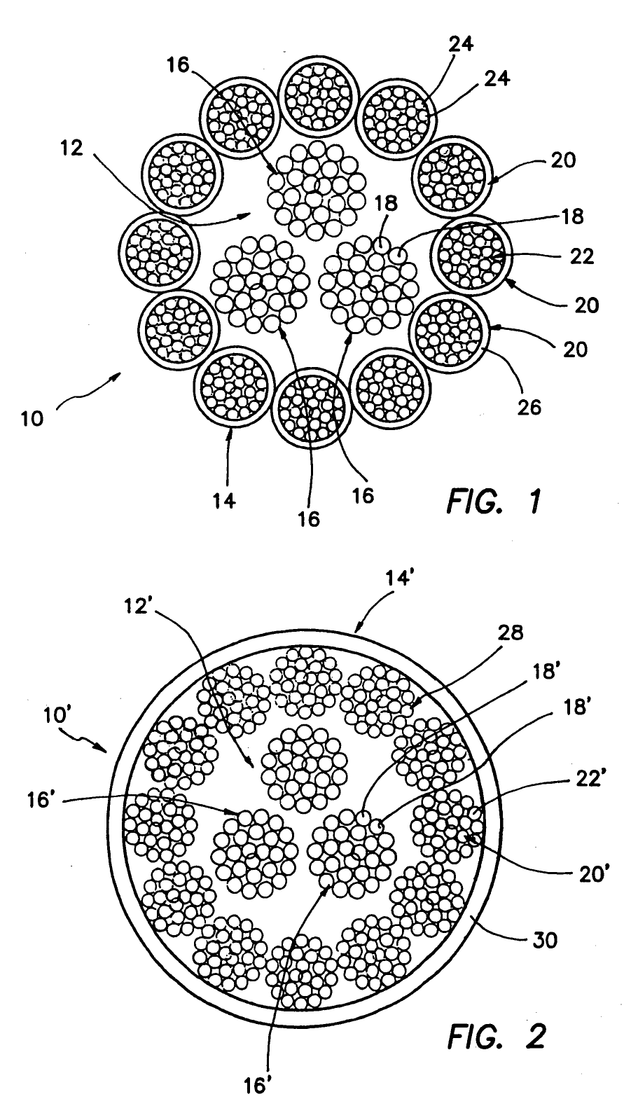

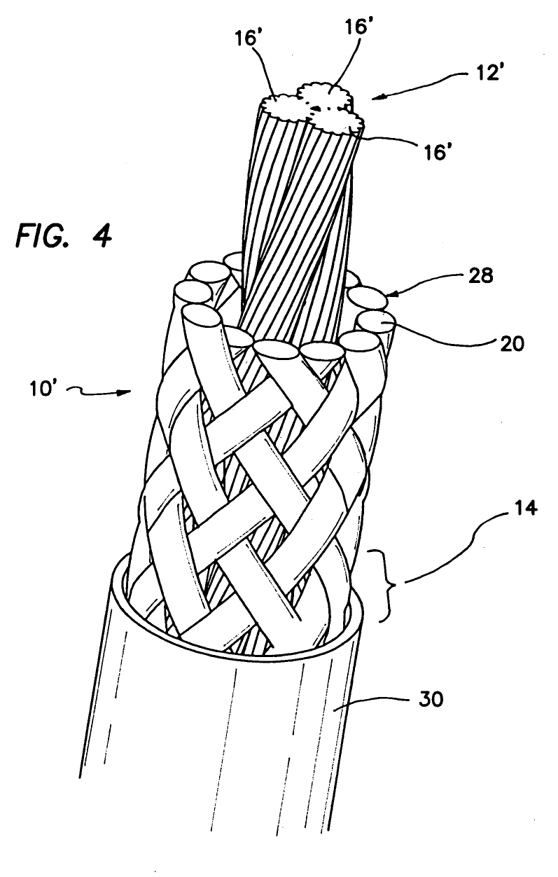

Referring now to FIGS. 1 and 3, a suture strand 10 according to the present invention comprises a core 12 and a cover 14. The core 12 comprises three core fibers 16 twisted together to form a single core element. Each core fiber 16 itself comprises a bundle of core filaments 18 of a first material. The cover 14 comprises an annular woven braid including eighteen cover fibers 20 surrounding the core 12. Each cover fiber 20 comprises a bundle of cover filaments 22 of a second material. Both the core filaments 18 and the cover filaments 22 are substantially circular in cross section, the core filaments 18 having larger diameters than the cover filaments 22. In addition, both the core fibers 16 and the cover fibers 20 are substantially circular in cross-section, the core fibers 16 having larger diameters than the cover fibers 20.

The first material, i.e. the material used to form the core filaments 18, is preferably a high strength, high molecular weight, high tenacity material such as ultrahigh molecular weight long chain polyethylene. The second material, i.e. the material used to form the cover filaments 24, is preferably a material having clinically acceptable knot tying characteristics. Preferably the second material is also opaque and available in a wide variety of colors. This allows the cover fibers 20 to be woven into a wide variety of visually distinguishable patterns, so the correct sutures can easily be identified in a multi-suture application. Acceptable materials having the proper combination of knot-tying ability and color selection include PET, polyester, coated urethanes, and mixtures thereof.

The size of the core 12 is selected according to the suture strength desired. An appropriately sized core 12 can increase the composite suture strength of a suture 10 having the construction of a standard #2 suture to that of a standard #5 suture.

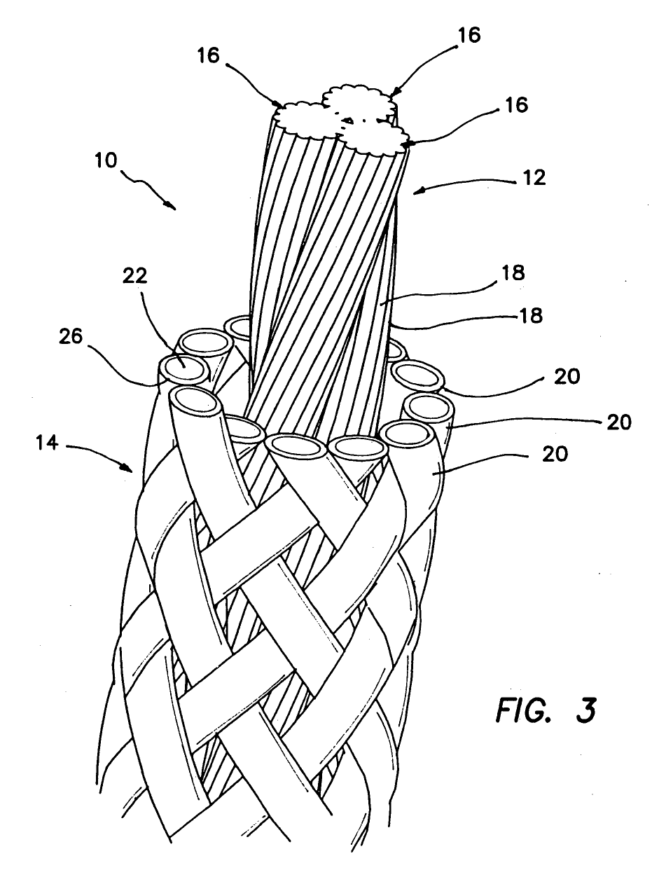

FIGS. 2 and 4 show a suture strand 10a according to an alternate embodiment of the invention. The core 12a comprises four core UHMWPE fibers 16a twisted together to form a single core element. The cover 14a comprises eighteen polyester cover fibers 20a woven together to form an annular braid around the core 12a. Each of the core fibers 16a is substantially wedge-shaped and has a length L that is greater than the diameter of each of the cover fibers 20a. The size of the core 12a is preferably selected to increase the increase the composite suture strength of a suture 10a having the construction of a standard #2 suture to that of a standard #5 suture.

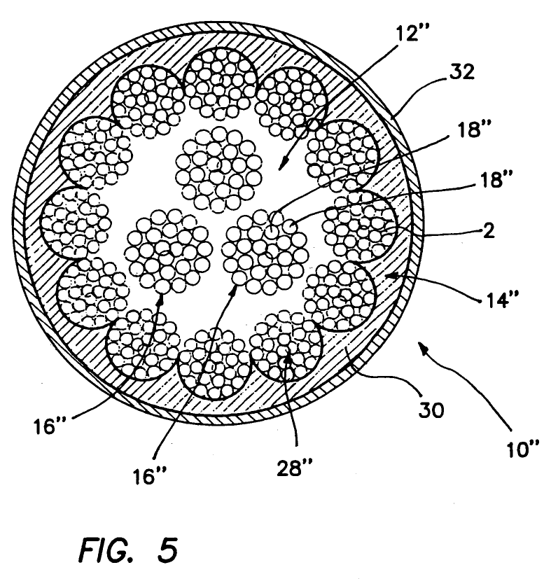

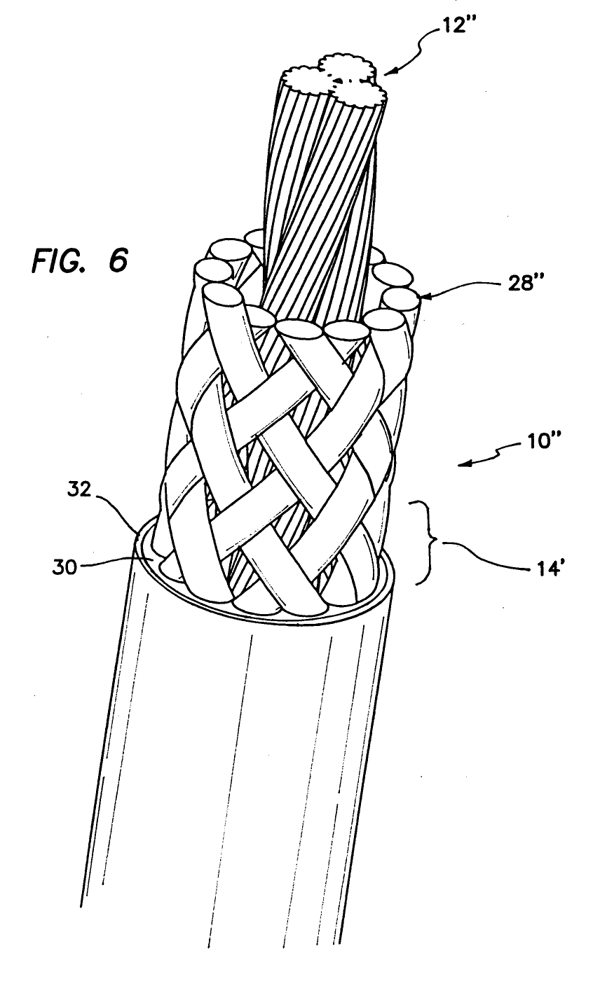

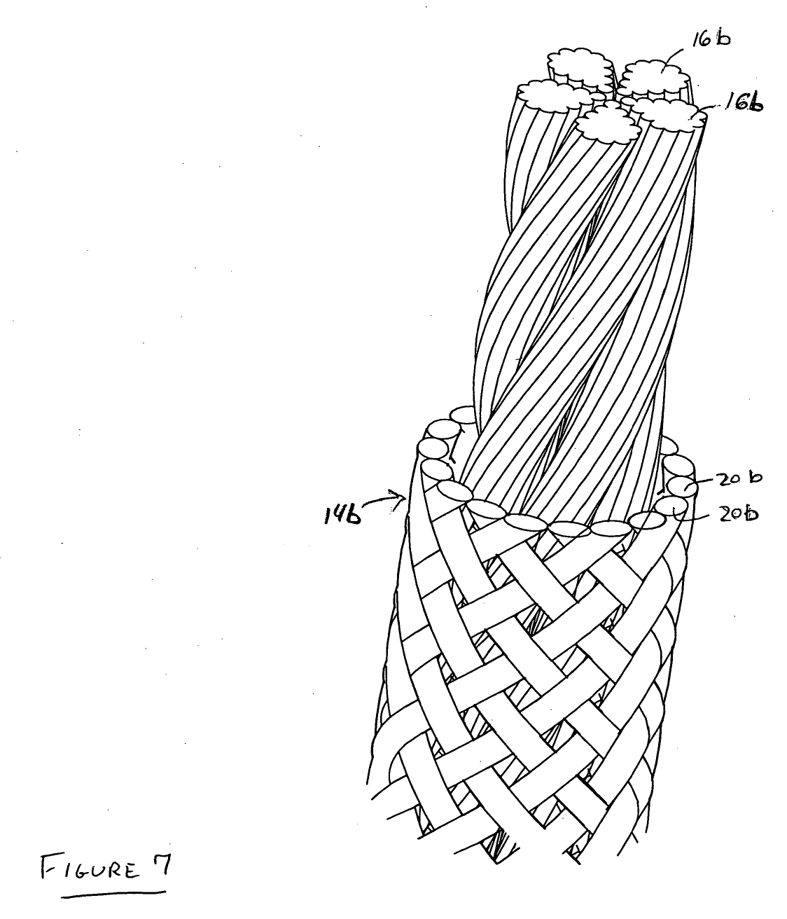

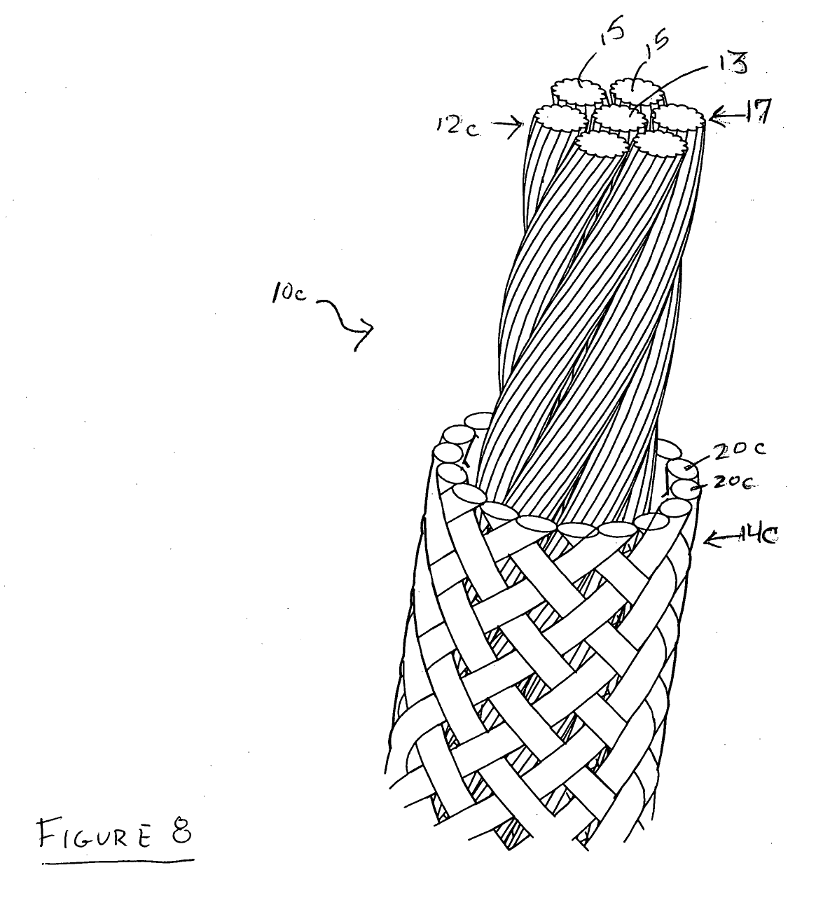

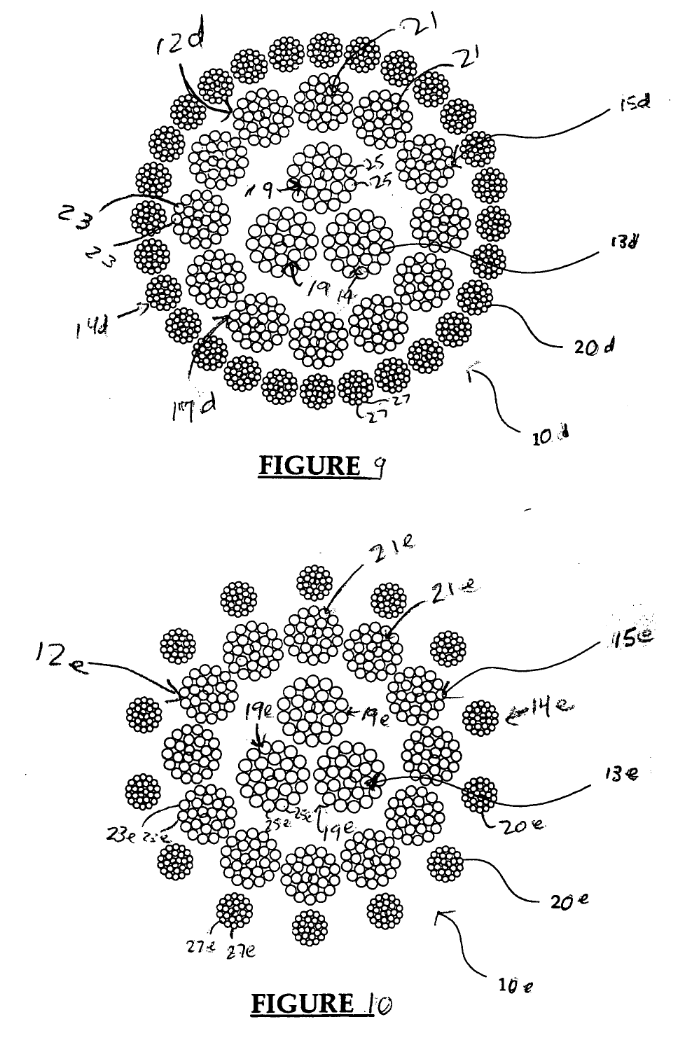

FIGS. 5 and 7 show a suture strand 10b according to another embodiment of the invention. The core 12b comprises five core UHMWPE fibers 16b twisted together to form a single core element. The cover 14b comprises eighteen polyester cover fibers 20b woven together to form an annular braid around the core 12b. Each of the core fibers 16b is substantially wedge-shaped and has a length L that is greater than the diameter of the cover fibers 20b. The size of the core 12b is preferably selected to increase the increase the composite suture strength of a suture 10b having the construction of a standard #2 suture to that of a standard #5 suture.

FIGS. 6 and 8 show a suture strand 10c according to still another embodiment of the invention. The core 12c comprises a sub-core 13 consisting of a single, centrally located UHMWPE sub-core fiber, surrounded by six UHMWPE core fibers 15 that have been woven or braided together to form an outer ring 17 around the sub-core 13. The cover 14c comprises eighteen polyester cover fibers 20c woven together to form an annular braid around the core 12c. This is a particularly flexible embodiment of the invention, having knot-tying characteristics similar to those of a standard #2 suture.

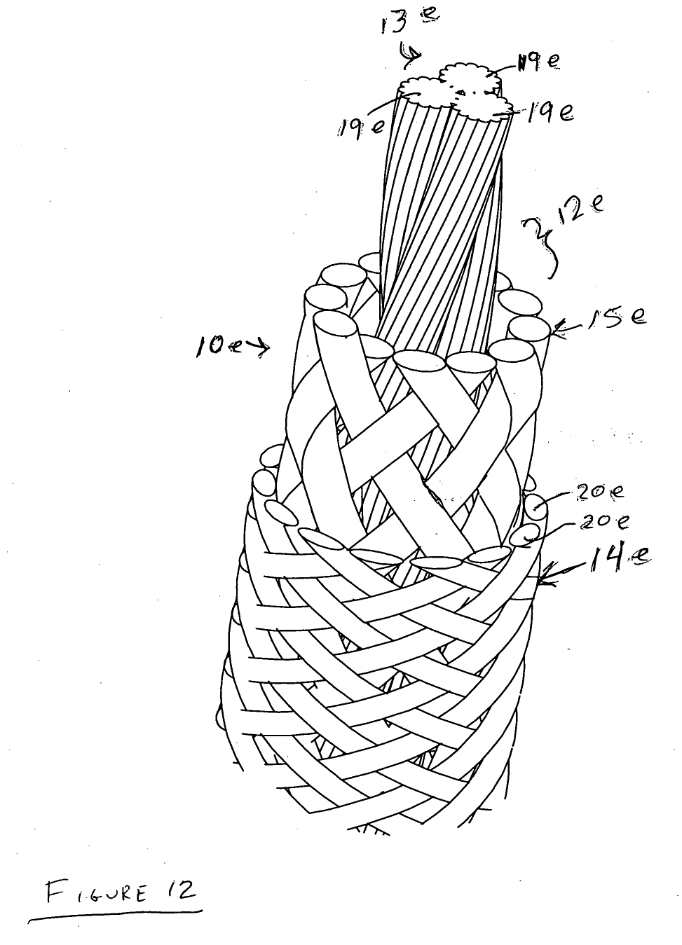

FIGS. 9 and 11 show a suture strand 10d according to yet another embodiment of the invention. The core 12d comprises a sub-core 13d consisting of three UHMWPE sub-core fibers 19 arranged in a twisted bundle, surrounded by twelve UHMWPE core fibers 21 that have been woven or braided together to form an outer ring 15d around the sub-core 13. The cover 14d comprises twenty eight polyester cover fibers 20d woven together to form an annular braid around the core 12d. The sub-core fibers 19, core fibers 21, and cover fibers 20d are all substantially circular in cross-section, the core fibers 21 in the outer ring 15d having diameters smaller than the diameters of the sub-core fibers 19 and larger than the diameters of the cover fibers 20d. In addition, the diameter of each core filament 23 within each core fiber 21 of the outer ring 15d is smaller than the diameter of each sub-core filament 25 within each sub-core fiber 19 and larger than the diameter of each cover filament 27 within each cover fiber 20d. This embodiment of the invention exhibits knot-tying characteristics identical to that of a standard #2 suture.

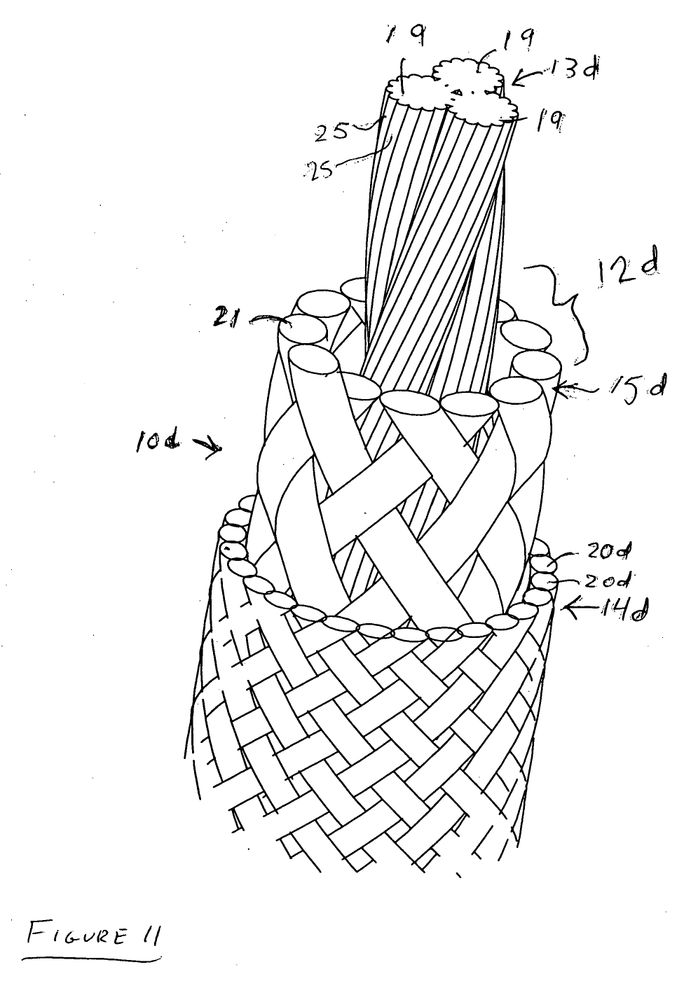

FIGS. 10 and 12 show a suture strand 10e according to yet another embodiment of the invention. The core 12e comprises a sub-core 13e consisting of three UHMWPE sub-core fibers 19e arranged in a twisted bundle, surrounded by twelve UHMWPE core fibers 21e that have been woven or braided together to form an outer ring 15e around the sub-core 13e. The cover 14e comprises fourteen polyester cover fibers 20e woven together to form an annular braid around the core 12e. The sub-core fibers 19e, core fibers 21e, and cover fibers 20e are all substantially circular in cross-section, the core fibers 21e in the outer ring 15e having diameters smaller than the diameters of the sub-core fibers 19e and larger than the diameters of the cover fibers 20e. In addition, the diameter of each core filament 23e within each core fiber 21e of the outer ring 15d is smaller than the diameter of each sub-core filament 25e within each sub-core fiber 19e and larger than the diameter of each cover filament 27e within each cover fiber 20e. This embodiment of the invention, like the previous embodiment, exhibits knot-tying characteristics identical to that of a standard #2 suture. Furthermore, the looser weave of the cover 14e increases the suture's coefficient of fiction, resulting in better knot-holding characteristics than the standard suture.

The suture of the present invention, which can be attached to a suture anchor or curved needle, is ideally suited for a wide variety of surgical procedures and in particular, most orthopedic procedures, including rotator cuff repair, Achilles tendon repair, patellar tendon repair, ACL/PCL reconstruction, hip and shoulder reconstruction procedures, and replacement of suture in anchors.

While this invention has been described with respect to various specific examples and embodiments, it is to be understood that the invention is not limited thereto and that it can be variously practiced within the scope of the following claims.

What is claimed is:

1. A suture strand suitable for use as a suture or ligature, comprising:
a core including a plurality of core fibers made of a first material; and
a cover surrounding the core, the cover including a plurality of cover fibers made of a second material different than the first material.

2. A suture strand according to claim 1, wherein the core fibers are arranged in a twisted bundle.

3. A suture strand according to claim 1, wherein the core consists solely of core fibers of the first material.

4. A suture strand according to claim 1, wherein the plurality of cover fibers are arranged to form a woven annular braid.

5. A suture strand according to claim 1, wherein the cover consists solely of cover fibers of the second material.

6. A suture strand according to claim 1, wherein the first material comprises a high tensile strength material.

7. A suture strand according to claim 1, wherein the first material is a high molecular weight, high tenacity material.

8. A suture strand according to claim 1, wherein the first material is ultrahigh molecular weight long chain polyethylene.

9. A suture strand according to claim 1, wherein the second material is a polymeric material selected from the group consisting of PET, polyester, coated urethanes, and mixtures thereof.

10. A suture strand according to claim 1, wherein the second material is substantially opaque.

11. A suture strand according to claim 1, wherein the first material is substantially transparent.

12. A suture strand according to claim 1, wherein each of the core fibers comprises a bundle of filaments of the first material.

13. A suture strand according to claim 1, wherein each of the cover fibers comprises a bundle of filaments of the second material.

14. A suture strand according to claim 1, wherein each of the core fibers is substantially circular in cross section.

15. A suture strand according to claim 1, wherein each of the core fibers is substantially wedge-shaped in cross-section.

16. A suture strand according to claim 1, wherein the core comprises:
a sub-core comprising at least one core fiber; and
an outer ring comprising a plurality of core fibers surrounding the sub-core.

17. A suture strand according to claim 16, wherein the sub-core comprises a single core fiber.

18. A suture strand according to claim 16, wherein the sub-core comprises a plurality of core fibers arranged in a twisted bundle.

19. A suture strand according to claim 16, wherein the outer ring comprises an annular woven braid of core fibers.

20. A suture strand according to claim 14, wherein:
each of the cover fibers is substantially circular in cross-section and has a first diameter; and
each of the core fibers has a diameter greater than the first diameter.

21. A suture strand according to claim 15, wherein:
each of the cover fibers is substantially circular in cross-section and has a first diameter; and
each of the core fibers has a length greater than the first diameter.

22. A suture strand according to claim 1, wherein:
each of the cover fibers comprises a bundle of cover filaments, each cover filament having a substantially circular cross-section and a cover filament diameter; and
each of the core fibers comprises a bundle of core filaments, each core filament having a substantially circular cross-section and a core filament diameter, wherein the core filament diameter of each of the core filaments is greater than the cover filament diameter of any of the cover filaments.

23. A suture strand suitable for use as a suture or ligature, comprising:
a core including a plurality of core fibers consisting solely of a first material; and
a cover surrounding the core, the cover including a plurality of cover fibers consisting solely of a second material different than the first material.

24. A suture strand according to claim 23, wherein the first material comprises a high tensile strength material.

25. A suture strand according to claim 23, wherein the first material is a high molecular weight, high tenacity material.

26. A suture strand according to claim 23, wherein the first material is ultrahigh molecular weight long chain polyethylene.

27. A suture strand according to claim 23, wherein the second material is a polymeric material selected from the group consisting of PET, polyester, coated urethanes, and mixtures thereof.

28. A suture strand according to claim 23, wherein the second material is substantially opaque.

29. A suture strand according to claim 23, wherein the first material is substantially transparent.

30. A suture strand according to claim 23, wherein the core comprises:
a sub-core comprising at least one core fiber; and
an outer ring comprising a plurality of core fibers surrounding the sub-core.

31. A suture strand according to claim 30, wherein the sub-core comprises a single core fiber.

32. A suture strand according to claim 30, wherein the sub-core comprises a plurality of core fibers arranged in a twisted bundle.

33. A suture strand according to claim 30, wherein the outer ring comprises an annular woven braid of core fibers.
